(19) United States
(12) Patent Application Publication
(10) Pub. No.: US 2004/0142351 A1
(43) Pub. Date: Jul. 22, 2004

(54) Associative analysis of gene expression array data

(75) Inventors: Igor Dozmorov, Oklahoma City, OK (US); Michael Centola, Oklahoma City, OK (US)

Correspondence Address: Michael C. Barrett, Esq., FULBRIGHT & JAWORSKI, L.L.P., Suite 2400, 600 Congress Avenue, Austin, TX, 78701, US

(73) Assignee: Oklahoma Medical Research Foundation

(21) Appl. No.: 10/693,656

(22) Filed: Oct. 24, 2003

Related U.S. Application Data
(60) Provisional application No. 60/420,826, filed on Oct. 24, 2002.

(51) Int. Cl. C12Q 1/68; G06F 19/00; G01N 33/48; G01N 33/50

(52) U.S. Cl. 435/6; 702/20

(57) ABSTRACT

Techniques for associative analysis of gene expression array data. A representative method includes collecting a plurality of expression profiles of a control group and an experimental group; normalizing the expression profiles relative to their backgrounds; adjusting the profiles to identify outliers and to re-scale to an averaged profile of the control group; identifying a group of similarly expressed genes, defining a reference group; identifying a plurality of differentially expressed genes in the experimental group using a paired T-test and an associative T-test; and classifying the differentially expressed genes as (a) likely false positive, (b) real positives, or (c) potential positives.

This application claims priority to, and incorporates by reference, U.S. Provisional Patent Application Serial No. 60/420,826, which was filed Oct. 24, 2002.

### STATEMENT AS TO RIGHTS TO INVENTIONS MADE UNDER FEDERALLY-SPONSORED RESEARCH AND DEVELOPMENT

This work was supported by grants from the National Institutes of Health National Center for Research Resources (grant numbers NIH 1 P20 RR15577 and NIH 1P20 RR16478), the Nathan Shock Center, and The Fund for Arthritis and Inflammatory Research (FAIR). The government may therefore own rights in the present invention.

### BACKGROUND OF THE INVENTION

1. Field of the Invention

The invention relates generally to the field of statistical analysis. More particularly, the invention relates to optimizing standards to guide statistical analysis. Even more particularly, the invention relates to optimizing standards to guide statistical analysis of gene expression.

2. Discussion of the Related Art

Analysis of data from large-scale mRNA expression studies is nontrivial due to the complexity and size of data sets and the fact that technical variation can be introduced at different stages in array production and processing. Establishing well specified and carefully validated procedures for standardization and normalization of data sets from individual specimens is a key first step in analysis, but no single method has proven free from ambiguity. Selection criteria based on the ratio of measured expression levels fails to account for intra-group variations (i.e. normal biologic variance) and can result in false positive selections (Kerr et al., 2000; Dozmorov et al., 2002). More progressive statistical approaches such as regression analysis, multidimensional scaling, or principal component analysis, have been cogently criticized on a number of grounds, including the influence of outliers (i.e. genes expressed to different degrees among samples), on the parameters of linear regression, principal axis choice, and the absence of information about variability of individual expression levels within homogenous groups of samples. Nonetheless, attempts of restricting the influence of outliers and non-correlated weak signals has not resulted in the development of recognized standards (Newton et al., 2001; Wu, 2001).

Additionally, current statistical methods do not adequately address the mutually exclusive characteristics of sensitivity and specificity. The common practice of using low thresholds for selection of significance (p<0.05) can also result in a large number of false positive selections. This is especially problematic for high-density arrays as the number of false positive selections expected to occur by chance may limit the ability to perform higher order analyses, such as molecular pathway identification or disease subphenotyping, that require groups of differentially expressed genes to be accurately predicted. Attempts to increase stringency by raising the threshold of significance above this value can also be problematic as it will cause a compensatory decrease in sensitivity and resultant increase in false negative selections. The use of large numbers of replicates is able to improve this situation (Glynne et al., 2000), although it can be expensive and labor intensive.

In earlier publications (Dozmorov et al., 2001; Dozmorov et al., 2002), normalization procedures have been applied to identification of differentially expressed genes in mice of the dwarf genotype (Ames—homozygous for the Prop 1^{df} mutation, and Snell—homozygous for the Pit1^{dw} mutation). Dwarf mice demonstrate similar deficiencies in pituitary dysfunction leading to decreased production of growth hormone, prolactin and thyroid-stimulating hormone and severe alterations in gene expression profiles relative to wild type mice (Pfaffle et aL, 1999). Here, for the first time, a full suite of useful statistical procedures is fully delineated.

### SUMMARY OF THE INVENTION

There is a need for the following embodiments. Of course, the invention is not limited to these embodiments.

In one embodiment, the invention involves a method of associative analysis. A plurality of expression profiles of a control group and a plurality of expression profiles of an experimental group are collected. The plurality of expression profiles of the control group are normalized relative to their backgrounds. The plurality of expression profiles of the experimental group are normalized relative to their backgrounds. The plurality of expression profiles of the control group and the plurality of expression profiles of the experimental group are adjusted to identify outliers and to re-scale to an averaged profile of the control group. A group of similarly expressed genes are identified, defining a reference group, determined from the plurality of expression profiles of the control group. A plurality of differentially expressed genes are identified in the plurality of expression profiles of the experimental group based on the reference group, wherein identifying the plurality of differentially expressed genes includes utilizing a paired T-test and an associative T-test. The differentially expressed genes are classified as (a) likely false positive, (b) real positives, or (c) potential positives using the paired T-test and associate T-test.

In other embodiments, identifying the plurality of differentially expressed genes further includes utilizing a Bonferroni T-test. Adjusting the plurality of expression profiles of the control group and the plurality of expression profiles of the experimental group can include: (a) selecting a plurality of genes from the plurality of expression profiles of the control group and the plurality of expression profiles of the experimental group, wherein the plurality of genes are expressed above a background; and (b) scaling the plurality of expression profiles of the control group and the plurality of expression profiles of the experimental group to an average profile of the plurality of expression profiles of the control group. Adjusting the plurality of expression profiles of the control group and the plurality of expression profiles of the experimental group can include analyzing by regression analysis the plurality of genes expressed above the background. Adjusting the plurality of expression profiles of the control group and the plurality of expression profiles of the experimental group can include selecting equally expressed genes as a homogenous family of genes with normally distributed residuals measured as deviations from a regression line that is calculated against an average profile.

The reference group can include a group of genes expressed above background levels with normal low variability of expression in control samples as determined by a F-test. The reference group can have residuals that approximate a normal distribution, based on a Kolmogorov-Smirnov criterion.

The associative T-test can include a test in which a plurality of replicated residuals for each gene of the plurality of the expression profiles of the experimental group are compared with an entire set of residuals from the plurality of expression profiles of the control group. The plurality of expression profiles of the control group and the plurality of expression profiles of the experimental group can include an array. Classifying can include: (a) classifying the genes identified as expressed by the paired T-test as false positive; (b) classifying the genes identified as expressed by the paired T-test and the associative T-test as real positives; and (c) classifying the genes identified as expressed by the associative T-test as potentially real positives. The genes identified as expressed by the associative T-test can be tested again. Identifying a group of similarly expressed genes determined from the plurality of expression profiles of the control group can further include excluding outliers from the plurality of expression profiles of the control group.

It will be understood that these, and other, embodiments, can be practiced by combining steps from different embodiments. These, and other, embodiments of the invention will be better appreciated and understood when considered in conjunction with the following description and the accompanying drawings.

### BRIEF DESCRIPTION OF THE DRAWINGS

The drawings accompanying and forming part of this specification are included to depict certain aspects of the invention.

FIGS. 1A-1C illustrate normalization of a gene expression profile to its own background.

FIG. 1A is a histogram showing the expression levels of 1176 cDNA targets derived from the liver of normal control mice. These values conform poorly to a normal distribution, with extended upper and lower tails apparent. Values from the lower tail result from background correction procedure and are typically negative. Values in the upper tail correspond to genes expressed above background in a given sample.

FIG. 1B shows a plot of the values with the proposed normal distribution versus the real levels of expression. The straight line is a regression line for the central part of the plot—predominantly background noise. To identify the parameters of normal distribution for background, data are sorted in ascending order and, as a first approximation, the mean and SD estimates are computed for all spots. Spots at the high end and at the low end of this distribution are then discarded one by one in alternating manner if they exceed a criterion set two SDs beyond the mean of the remainder of the distribution. The resulting set of nondiscarded points (typically between 500 and 600 of the initial set of 1176) represents the fragment of normally distributed background values.

FIG. 1C shows that the fragment is then used for the accurate estimation of the parameters of the normal distribution for background using a standard minimization procedure. The mean and SD of normally distributed background spots are used for the raw intensity S normalization as S′=(S−Av)/SD. The distribution of S′ (FIG. 1D) has a mean of zero and SD=1 over the set of background genes. The curve shows the distribution of these non-expressed genes. The threshold 3SD =3 was used for selection of genes expressed above background.

FIGS. 2A and 2B illustrate a comparison of liver samples of two normal mice (Atlas I arrays as in FIG. 1C). Each data set (S1 and S2) has been normalized with respect to its own set of background genes, as explained above. The values are shown on a logarithmic scale, and only “expressed above background” values where S>Log(3) are included. Differentially expressed genes can be identified as those whose ratio of expression in two control samples does not fall on or close to the line describing similarly expressed genes (filled circles in FIG. 2A). These genes denoted as “outliers” were excluded from resealing by use of a robust regression procedure in which the influence of outliers is down-weighted in a series of regression procedures using NCSS STAT SYSTEM (Number Cruncher Statistical System, Utah, 2001) with an influence function based on the use of least absolute deviations and with twenty subsequent cycles of the regression parameters estimations. FIG. 2B shows the resulting plot for completely adjusted distributions with the final regression line passing through the origin with the slope equal 45°.

FIGS. 3A-3C illustrate deviations of gene expression after rescaling to the averaged data in normal mice group (8 mice).

FIG. 3A shows a variability of genes within the homogenous control group (the residuals were calculated as differences between gene expression in each control sample and its average).

FIG. 3B shows the same data after exclusion of hyper-variable genes with a SD statistically higher than the homogeneous control group (based on an F-criterion).

FIG. 3C shows a deviation from normal control averages of gene expressions in dwarf mice samples.

FIG. 4 illustrates a sensitivity and specificity of statistical comparison. Numbers of genes with statistically different expression in dwarf mice compared with their normal siblings were selected from 256 expressed genes presented on Atlas-I membrane using three different criterions—paired T-test (p<0.05), Bonferroni T-test (p<0.05/256), and associative T-test (p<0.0025). Positive, false positive and false negative selections shown with different filling as indicated.

### DETAILED DESCRIPTION

Embodiments of the invention and the various features and advantageous details of those embodiments are explained more fully with reference to the nonlimiting embodiments that are illustrated in the accompanying drawings and detailed in the following description.

Methods

Statistical methods of comparative analysis of cDNA array data are described here and claimed in a novel manner. The method, denoted “associative analysis,” supplements the standard procedure of multiple paired comparisons by associating the expression level of each gene in an experimental group with a family of similarly and stably expressed genes in a control group. This associative analysis enhances the sensitivity of selections beyond previously described modifications of the T-test and increases the number of differentially expressed genes identified without significantly increasing the misidentification of false positives.

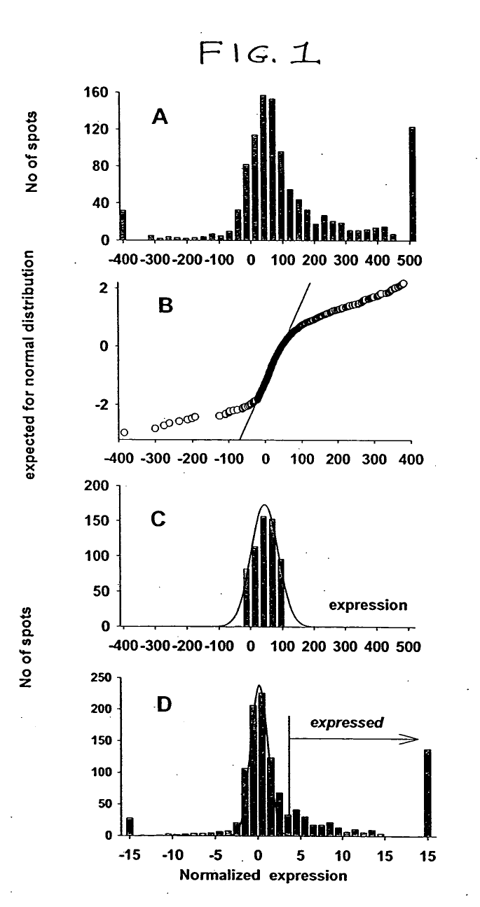

In one embodiment, the analysis starts by normalizing each expression profile to its own background, with selection of the genes expressed above background for subsequent adjustment and comparison. The expressed genes are selected as not being associated with a representative homogenous family of background level values having normal distribution (FIG. 1).

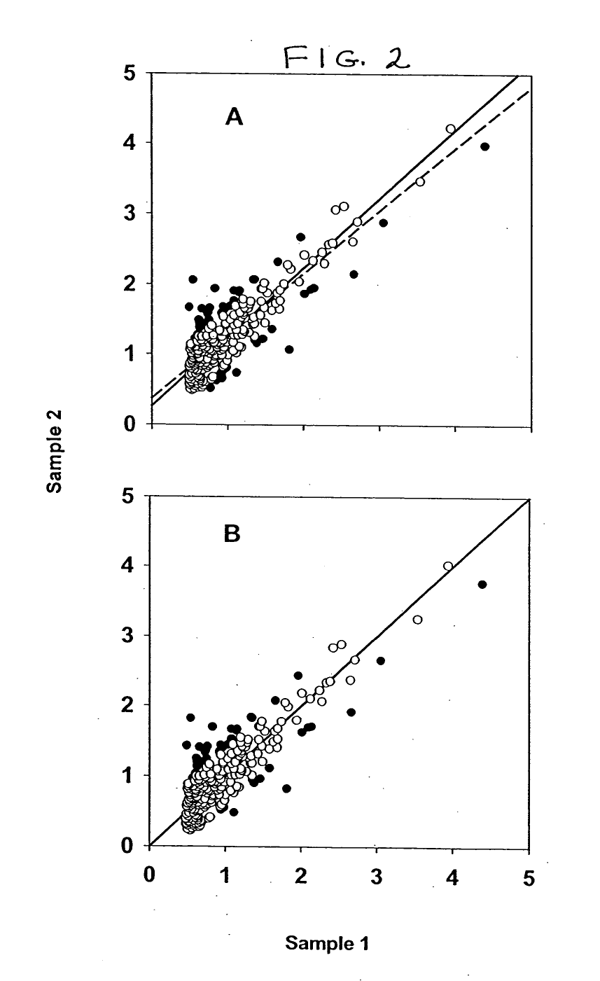

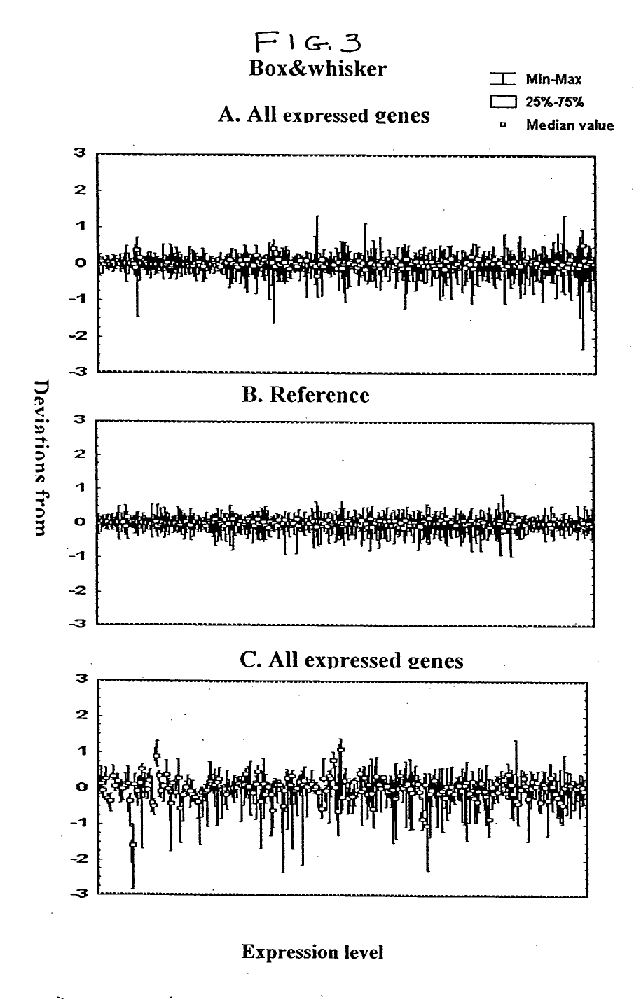

The normalized profiles may then be adjusted relative to each other by robust regression analysis of genes expressed above background. In this analysis, potential outliers are identified and their contribution to the calculations down-weighted in an iterative manner, diminishing or excluding their influence (FIG. 2). Expression profiles of both control and experimental groups are then re-scaled to a common standard—the averaged profile of the control group. An alternative procedure for outliers exclusion may be based on the selection of equally expressed genes as homogenous family of genes with normally distributed residuals measured as deviations from the regression line calculated against the averaged profile (FIG. 3). Outliers may thereafter be determined as having deviations not associated with this normal distribution presented by several hundred members.

After the profiles have been adjusted, a group of similarly expressed genes from control experiments, denoted “reference group” (FIG. 3), to be used for statistical analysis of differentially expressed genes using an associative T-test, is identified. The reference group is composed of a group of genes expressed above background levels with normal low variability of expression in control samples as determined by an F-test, and whose residuals may approximate a normal distribution, based on the Kolmogorov-Smirnov criterion.

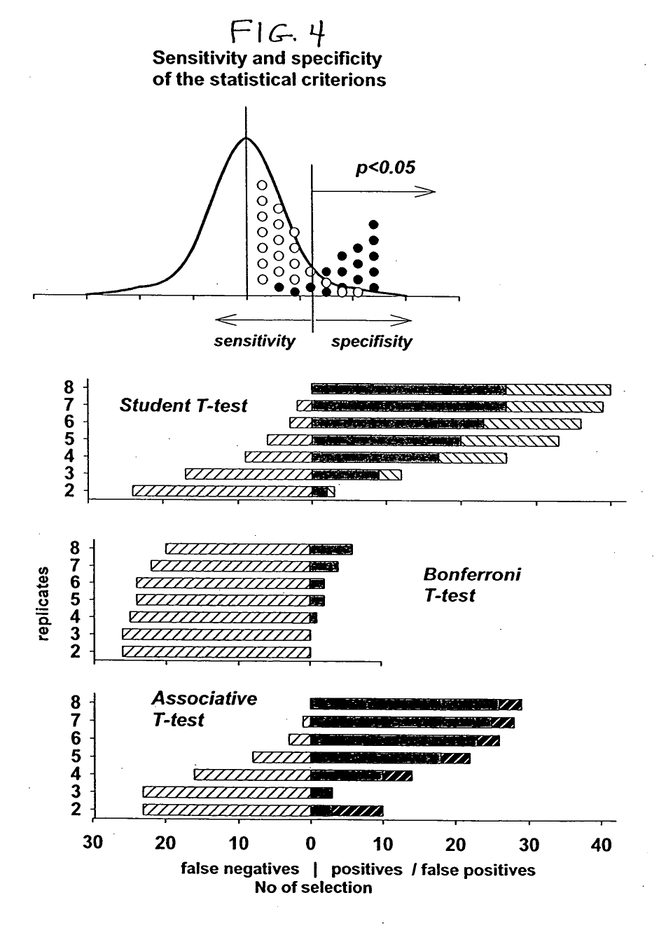

Genes differentially expressed in experimental versus control groups can then be identified using distinct statistical approaches (FIG. 4). These approaches are described below.

(a) A paired T-test, which selects differentially expressed genes, (separate tests for a pair of replicates of each gene in the control and experimental groups) and the commonly accepted significance threshold of p<0.05. A significant proportion of the genes identified as differentially expressed will be false positive determinations at this threshold level.

(b) A T-test using a Bonferroni correction for the significance threshold that may eliminate false positive determinations with simultaneous loss of the sensitivity, and resulting in increased proportion of false negative determinations.

(c) An associative T-test in which the replicated residuals for each gene of the experimental group are compared with the entire set of residuals from the reference group defined earlier. The null hypothesis is checked to determine if gene expression in the experimental group is associated with the reference group defined above. The significance threshold is corrected to make improbable the appearance of false positive determinations.

(d) Comparing the selections from the paired T-test and associative T-tests to classify the differentially expressed genes as: (a) likely false positives (these are genes selected as differentially expressed by the paired T-test with p<0.05, but not by the associative T-test); (b) real positives (selected in both tests) (c) potential positives (genes selected in the associative test only).

Results

Comparative analysis of gene expressions in the experimental group is begun by applying the procedures of normalization to background and rescaling described above. Averaged data from the control group is used as a standard for data rescaling. The adjustment of data from the experimental group to averaged control data will produce the same order residuals for equally expressed genes and highlight the genes with extreme expression deviations (FIG. 3C).

Single Gene Comparisons—Paired T-Test

The paired T-test evaluates the difference between the means of each single gene expression in two groups employing the variance within groups as an error term. The use of the usual threshold p=0.05 for the selection of differentially expressed genes may result in a significant proportion of false positive selections from experiments with thousands of elements, as is the case in array experiments. When using the Atlas 1.2 array set, about 50 false positive selections can be expected at this threshold if all genes are analyzed. This number can be substantially decreased if the analysis excludes genes that are not expressed in both groups. Approximately 250 genes were determined to be expressed in the experiments described here. The proportion of false positive determinations expected in this group at p=0.05, which is 12 to 13, may represent a significant portion of the total number of differentially expressed genes identified. Use of replicates may result in a decrease of the proportion of false negative determinations though the proportion of false positives remains relative stable—around one third of all positive selections (FIGS. 4A and 4B). This proportion may be decreased through the use of a corrected p value.

Single Gene Comparison Bonferroni T-Test

The Bonferroni correction may be employed to reduce the proportion of false positive determinations in multiple comparison analysis, and it may be applied to array data. In this method the stringency of the threshold p is increased to 0.05/(the number of compared values). For the expressed genes identified above, p is equal to 2×10⁻⁴ (p=0.05/250=2×10⁻⁴). This increased threshold produces a new selection of differentially expressed genes with the absence of false positive determinations (FIG. 4C). While specificity is increased in this analysis, sensitivity is sacrificed and a large number of false negatives, type II errors, are obtained. All selections obtained with Bonferroni T-test are present also within selections made in Associative comparison.

Associative Comparison

It is possible to substitute the typical paired comparison of gene expressions between control and experimental groups with the comparison of their residuals. In this analysis it is determined if a given gene of the experimental group belongs to (or can be associated with) the reference group. Denoted an associative T test, it is actually a standard Student T-test applied to the comparison of expression deviations. An associative T-test dramatically increases the power of comparisons relative to a paired T-test. In the data analyzed here, this is due to the fact that eight replicates from the control group are compared with several hundred values of the reference group. As a result, a large number of positive determinations can be obtained with stringent thresholds (FIG. 4D).

By comparing the results of these two tests, paired T-test with threshold p<0.05, and associative T-test with threshold p<0.005 (p<1/n, where n=number of genes analyzed from the experimental group), differentially expressed genes can be classified into three groups. Genes defined as differentially expressed by the paired T-test but not by the associative T-test are likely false positives. Genes identified in both analyses are likely real positives, that also include the small sub-group of genes selected by the Bonferroni T-test. Genes identified in the associative analyses are potentially real positives that require additional replicates to confirm.

This analysis has been used to identify genes that are differentially expressed between normal and dwarf mice and found 46 genes overexpressed in Snell dwarf mice; 49 genes expressed only in Snell mice; 12 genes overexpressed in normal control mice; 13 genes expressed only in normal mice (Table 1A-1D in the Appendix). Of these selected genes, 71 are previously reported as differentially expressed in Snell dwarf mice, associated with dwarfism, or strongly associated with a similar hormonal status. An additional 10 selections obtained by the new method and not obtained by previous analysis, whose relevance to dwarfism or similar hormonal status are supported by the indicated references, are listed in Table 2. Only genes that passed both criterions (a) standard paired T test (sT-test) with threshold p<0.05; and (b) associative T-test (aT-test) with threshold p<0.005; are presented in Table 2. In addition, this new method was able to more correctly predict the expression levels of 11 genes verified by RT-PCR.

TABLE 2

| Gene Name | Relationship to dwarfism |
|---|---|
| Ets-related transcription | Synergistic interaction of Pit-1 with a |
| factor; E74-like factor 1 | member of the Ets family of |
| | transcription factors |
| Insulin-like growth factor | Transcript is elevated in dwarf rodents. |
| binding protein 2 precursor | |
| Serine protease inhibitor 2.2 | A growth hormone regulated serine |
| | protease inhibitor. |
| Phosphoglycerate kinase 1 | Transgenic mice with transgene under |
| | the control of the mouse phosphoglycerate |
| | kinase gene, selectively expresses |
| | GHRH-RP (GH-releasing hormone |
| | related peptide), but not GHRH. |
| Transducer of erbB2 | Under GH control |
| Growth hormone releasing | Not expressed in dw/dw mice |
| hormone | |
| Ceruloplasmin | Low level in patients with GH deficiency |
| Phosphodiesterase I | Increased in Snell mice |
| IGF binding protein/receptor | Increased in dwarf mice |
| Glutathione S-transferase | Increased in dwarf mice |
| alpha 2 | |

Discussion

Useful and practical multistep procedures to analyze gene expression data from a cDNA array are described in this disclosure. The techniques provide a robust means of normalizing one channel data using an internal standard; establish a more precise procedure for data scaling by reducing the influence of outliers upon calculation of scalars; increase the sensitivity of differential gene identification without loss of specificity; and allow differentially expressed genes to be classified into distinct groups of probabilistically known or suspected differential expression.

An opportunity to increase the power of statistical analysis using representative standards for selection of potential outliers is presented here. This general procedure is done three times in these analyses. The first representative standard is the family of genes whose hybridization signals are at or below the background level. Outliers from this standard are defined as “expressed genes.” The second representative standard is the family of normally distributed residuals of equally expressed genes of the control group. Outliers from this group are hypervariable and differentially expressed genes that must be excluded from regression analysis for proper adjustment of pairs of profiles under comparison. The third representative standard is the family of genes with low variability within replicate control samples. There are two types of outliers from this standard—hypervariable genes of the control group (which were excluded to create this standard) and differentially expressed genes of the experimental group—whose identification is the main goal of these analyses.

The necessity to initially exclude from comparisons expressed from non-expressed genes was demonstrated here with data obtained from Snell mice using Clontech Atlas (Clontech, San Diego, Calif.) arrays in which 600 genes were spotted in duplicate. Since two independent signals are measured for each gene on the membrane, the variation in intensity between the duplicated spots for a given gene can be used to assess signal reproducibility. If variation were independent of signal intensity, the ratio of variation between duplicate spots would be distributed around 1 with small random variations. However, this was not observed for genes expressed below some threshold signal intensity. It is of note that this threshold corresponds to the determination of background. This so called background threshold may be due to technical limitations of measuring signal intensity on the array or it may be a real biologic threshold defined by genes that are not expressed. Operationally however, the addition of this exclusion criterion provides a logical cutoff between noncorrelative and correlative data and therefore improves the reliability of the comparative analysis carried on after this step. While these exclusion criteria improve the homogeneity of selections made using ratios, the arbitrariness can be associated with loss of useful information about low abundance genes that can play an important role in regulatory biological processes.

Further enrichment of reliability on signal variation is also accomplished. There are different sources for fluctuations in residuals. Technological variations represent a random component of deviation and are therefore common for all expressions. Some publications demonstrate the dependence of technological fluctuations on the level of gene expression, and a resultant non-normal distribution of these values. The two main sources of heterogeneity in gene expression variations are the “additive component,” prominent at low expression levels, and the “multiplicative component,” prominent at high expression levels. The intensity measurement yᵢⱼ for gene i [symbol] I={i₁, . . . , iₙ} in sample j [symbol] J={j₁, . . . , jₘ} is modeled by the equation yᵢⱼ=αᵢⱼ+μᵢⱼ×e⁷²+εᵢⱼ, where α is the normal background (and independent of expression level), μ is the expression level in arbitrary units, ε is first error term (additive) which represents the standard deviation of background, and η is the second error term, which represents the proportional error (multiplicative). The first error term is excluded in the analysis by eliminating expression values at or below background levels. The second error term is transformed from multiplicative (and therefore expression-dependent, increasing in proportion to expression level), into additive or expression independent) by log-transformation of data: log(y)=log(μ)+η, where η is the residual for log-transformed data. The independence of η from individual gene expressions is proven by the vendor (Atlas manual, 2000) and confirmed with the Kolmogorov-Smimov normality test in the experiments.

It has been found that the number of repetitions can be critical in achieving adequate specificity (low false positives) and sensitivity (low false negatives). Due to the stochastic character of the above-mentioned fluctuations, replication and averaging is a sensible method to reduce the noise level. Only those transcripts that are truly altered by an experimental factor will have a reproducible change and become more statistically significant with repetition; those changes that result from noise will not become more significant with repetition. Thus, sensitivity increases with repetition at a fixed specificity.

Both the paired T-test and the associative T-test demonstrate similar improvement in sensitivity through replication. However, the specificity of paired T-test remains unchanged when using from 4 to 8 replicates. This is due to the use of the necessity to use conservative methods to protect from false positive determinations when using the Paired T-test. These methods result in the loss of information about the majority of false negative expression differences. This information, once lost, is not regained through additional replicates. In the associative T-test, selections are made at a significance threshold high enough to exclude the appearance of false positive determinations. However, the number of comparisons made between a given experimental gene and the family of similarly expressed genes in the control condition assures that few false negative determinations will occur. Increased repetition can therefore be used to enhance the overall statistical significance of the selections made using this method (FIG. 4).

Conformation of the increased sensitivity of this method was obtained from a literature search of genes whose expression has been shown to be different in Snell mice and related model systems. At the level of sensitivity with less than one false positive determination the associative method selects a larger number of differentially expressed genes documented in the literature to have links with dwarfism or similar abnormalities in hormonal status than previous methods utilizing a paired analysis. Only half (approximately 30) of the differences obtained by microarray studies that utilized a standard paired analysis (Dozmorov et al., 2002) were confirmed in the current analysis. Importantly, only those genes confirmed by the associative method have been shown to be related with a premature aging phenotype in empirical studies, suggesting the methods described here do indeed increase the specificity of differential gene identification.

The associative method also enhances the information obtained from microarray experiments beyond common approaches because it discriminates between genes that are differential expressed from those that are expressed only in one state. For example Calgranulin B has been shown previously by RT-PCR to be undetectable in normal mice, as predicted by the method described herein, yet selected as differentially expressed in a previous analysis utilizing only a standard paired comparison (Dozmorov et al., 2002).

By testing the hypothesis of association of any potential outlier with a large representative standard, typically several hundreds elements, the statistical power of the analysis is increased over that achieved with traditional single gene comparisons which are powered only by the numbers of replicates. The higher power of the associative test, thus, increases sensitivity without loss of specificity. When used in combination with a traditional paired analysis, this increased statistical power also allows the use of traditional low level significance cutoffs in the standard paired analysis (p<0.05) without the risk of including false positive selections. The associative analysis is therefore based on an idea opposite to the commonly held view that large-scale array experiments suffer from compensatory tradeoffs in sensitivity and specificity. In fact, the procedures presented here demonstrate that large-scale data sets are information-rich and provide a means for discriminating common technical variation from individual biological variability.

The terms a or an, as used herein, are defined as one or more than one. The term plurality, as used herein, is defined as two or more than two. The term another, as used herein, is defined as at least a second or more. The terms including and/or having, as used herein, are defined as comprising (i.e., open language). The term approximately, as used herein, is defined as at least close to a given value (e.g., preferably within 10% of, more preferably within 1% of, and most preferably within 0.1% of).

### Practical Applications and Advantages of the Invention

A practical application of the invention that has value within the technological arts is an analysis of data sets, such as identifying molecular pathways or classifying disease subphenotypes. There are virtually innumerable other uses for the invention, which will be recognized by one having ordinary skill in the art.

Variation may be made in the steps or in the sequence of steps composing methods described here.

The appended claims are not to be interpreted as including means-plus-function limitations, unless such a limitation is explicitly recited in a given claim using the phrase(s) “means for” and/or “step for.” Subgeneric embodiments of the invention may be delineated by the appended independent claims and their equivalents. Specific embodiments of the invention may be differentiated by the appended dependent claims and their equivalents.

### References

Each of the references listed below is incorporated by reference in its entirety.

1. Clontech Atlas cDNA expression arrays user manual. PT3140-1, October 2000.

2. Dozmorov, I., A. Bartke, and R. A. Miller (2001). Array-based expression analysis of mouse liver genes: Effect of age and of the longevity mutant Propldf. J Gerontol 56A, B72-B80.

3. Dozmorov, I., A. Galecki, Y. Chang, R. Krzesicki, M. Vergara, and R.A. Miller (2002). Gene expression profile of long-lived Snell dwarf mice. J Gerontol 57A, B 1-B10.

4. Glynne, R. J., G. Ghandour, and C. C. Goodnow (2000). Genomic-scale gene expression analysis of lymphocyte growth, tolerance and malignancy. Curr Opin Iruunol 12(2), 210-4.

5. Kerr, M. K., M. Martin, and G. A. Churchill (2000). Analysis of variance for gene expression microarray data. J Comput Biol 7(6), 819-37.

6. Khan, J., J. S. Wei, M. Ringner, L. H. Saal, M. Ladanyi, F. Westermann, F. Berthold, M. Schwab, C. R. Antonescu, C. Peterson, and P. S. Meltzer (2001) Classification and diagnostic prediction of cancers using gene expression profiling and artificial neural networks. NatureMed 7(6),673-9

7. Lee, M. L., F. C. Kuo, G. A. Whitmore, and J. Sklar (2000) Importance of replication in microarray gene expression studies: statistical methods and evidence from repetitive cDNA hybridizations. Proc Natl Acad Sci USA 97 (18), 9834-9.

8. Miller, R. A., A. Galecki, and R. J. Shmookler-Reis (2001) Interpretation, design, and analysis of gene array expression experiments. J Gerontol 56A, B52-B57.

9. Newton, M. A., C. M. Kendziorski, C. S. Richmond, F. R. Blattner, and K. W. Tsui (2001). On differential variability of expression ratios: improving statistical inference about gene expression changes from microarray data. J Comput Biol 8(1), 37-52.

10. Pfaffle, R. W., O. Blankenstein, S. Wuller, and H. Kentrup (1999). Combined pituitary hormone deficiency: role of Pit-1 and Prop-1. Acta Paediatr Suppl 88(433), 33-41.

11. Rocke, D. M., and B. A. Durbin (2001). A model for measurement error for gene expression Analysis. J Comput Biol 8(6), 557-569.

12. Wittes, J., and H. P. Friedman (1999). Searching for evidence of altered gene expression: A comment on statistical analysis of microarray data. J Nat Cancer Inst 91, 400-401

13. Wu, T. D. (2001). Analysing gene expression data from DNA microarrays to identify candidate genes. J Pathol 195(1), 53-65.

14. Yang, M. C., Q. G. Ruan, J. J. Yang, S. Eckenrode, S. Wu, R. A. Mclndoe, and J. X. She (2001). A statistical method for flagging weak spots improves normalization and ratio estimates in microarrays. Physiol Genomics 7(1),45-53

15. Zien, A., T. Aigner, R. Zimmer, and T. Lengauer (2001) Centralization: A new method for the normalization of gene expression data. Bioinformatics 1, 1-9

1. A method of associative analysis, comprising:
collecting a plurality of expression profiles of a control group and a plurality of expression profiles of an experimental group;
normalizing the plurality of expression profiles of the control group relative to their backgrounds;
normalizing the plurality of expression profiles of the experimental group relative to their backgrounds;
adjusting the plurality of expression profiles of the control group and the plurality of expression profiles of the experimental group to identify outliers and to re-scale to an averaged profile of the control group;
identifying a group of similarly expressed genes, defining a reference group, determined from the plurality of expression profiles of the control group;
identifying a plurality of differentially expressed genes in the plurality of expression profiles of the experimental group based on the reference group, wherein identifying the plurality of differentially expressed genes comprises utilizing a paired T-test and an associative T-test; and
classifying the differentially expressed genes as (a) likely false positive, (b) real positives, or (c) potential positives using the paired T-test and associate T-test.

2. The method of claim 1, wherein identifying the plurality of differentially expressed genes further comprises utilizing a Bonferroni T-test.

3. The method of claim 1, wherein adjusting the plurality of expression profiles of the control group and the plurality of expression profiles of the experimental group comprises:
selecting a plurality of genes from the plurality of expression profiles of the control group and the plurality of expression profiles of the experimental group, wherein the plurality of genes are expressed above a background; and
scaling the plurality of expression profiles of the control group and the plurality of expression profiles of the experimental group to an average profile of the plurality of expression profiles of the control group.

4. The method of claim 1, wherein adjusting the plurality of expression profiles of the control group and the plurality of expression profiles of the experimental group comprises analyzing by regression analysis the plurality of genes expressed above the background.

5. The method of claim 1, wherein adjusting the plurality of expression profiles of the control group and the plurality of expression profiles of the experimental group comprises selecting equally expressed genes as a homogenous family of genes with normally distributed residuals measured as deviations from a regression line that is calculated against an average profile.

6. The method of claim 1, wherein the reference group comprises a group of genes expressed above background levels with normal low variability of expression in control samples as determined by a F-test.

7. The method of claim 1, wherein the reference group has residuals that approximate a normal distribution, based on a Kolmogorov-Smimov criterion.

8. The method of claim 1, wherein the associative T-test comprises a test in which a plurality of replicated residuals for each gene of the plurality of the expression profiles of the experimental group are compared with an entire set of residuals from the plurality of expression profiles of the control group.

9. The method of claim 1, wherein the plurality of expression profiles of the control group and the plurality of expression profiles of the experimental group comprise an array.

10. The method of claim 1, wherein classifying comprises:
classifying the genes identified as expressed by the paired T-test as false positive;
classifying the genes identified as expressed by the paired T-test and the associative T-test as-real positives; and
classifying the genes identified as expressed by the associative T-test as potentially real positives.

11. The method of claim 10, wherein the genes identified as expressed by the associative T-test are tested again.

12. The method of claim 1, wherein identifying a group of similarly expressed genes determined from the plurality of expression profiles of the control group further comprises excluding outliers from the plurality of expression profiles of the control group.

13. A method of associative analysis, comprising:
collecting a plurality of expression profiles of a control group and a plurality of expression profiles of an experimental group;
normalizing the plurality of expression profiles of the control group relative to their backgrounds;
nonnalizing the plurality of expression profiles of the experimental group relative to their backgrounds;
adjusting the plurality of expression profiles of the control group and the plurality of expression profiles of the experimental group to identify outliers and to re-scale to an averaged profile of the control group;
identifying a group of similarly expressed genes, defining a reference group, determined from the plurality of expression profiles of the control group, the reference group comprising a group of genes expressed above background levels with normal low variability of expression in control samples as determined by a F-test;
identifying a plurality of differentially expressed genes in the plurality of expression profiles of the experimental group based on the reference group, wherein identifying the plurality of differentially expressed genes comprises utilizing a paired T-test, a Bonferroni T-test, and an associative T-test;
classifying the genes identified as expressed by the paired T-test as false positive;
classifying the genes identified as expressed by the paired T-test and the associative T-test as real positives; and
classifying the genes identified as expressed by the associative T-test as potentially real positives.
